(19) United States
(12) Patent Application Publication
(10) Pub. No.: US 2006/0051309 A1
(43) Pub. Date: Mar. 9, 2006

(54) Cosmetic dressing containing a waxy cmponent, a film-forming agent and a gel-forming substance

(76) Inventors: Maike Patzer, Aurachtal (DE); Azra Swistowski, Nurmberg (DE); Christina Zech, Kaufering (DE)

Correspondence Address: BACHMAN & LAPOINTE, P.C., 900 CHAPEL STREET, SUITE 1201, NEW HAVEN, CT, 06510, US

(21) Appl. No.: 10/534,686

(22) PCT Filed: Nov. 11, 2003
(86) PCT No.: PCT/EP03/12595

§ 371 Date: Jul. 28, 2005

(30) Foreign Application Priority Data
Nov. 13, 2002 (DE) 102 52 816.0

(51) Int. Cl. A61K 8/81; A61K 8/73

(52) U.S. Cl. 424/70.13; 424/70.16; 424/70.17

(57) ABSTRACT

A cosmetic preparation in the form of an O/W emulsion which, besides usual cosmetic ingredients, contains a wax component, a film-forming system and at least one gel component, wherein the gel component has at least one swollen hydrocolloid.

### BACKGROUND OF THE INVENTION

The invention concerns a cosmetic preparation, in particular a preparation for coloring and forming keratinic material, and a process for the production thereof and the use thereof.

Cosmetic preparations with which keratinic material, for example eyelashes, can be colored, have already long been known. In order to do justice to the many different requirements which are to be made on such a preparation, improvements have been repeatedly developed. Compositions which are applied to eyelashes are intended on the one hand to provide for good coloring of the eyelashes, but they should not come off on to the surrounding area. They should adhere well to the eyelashes, but they should also be easy to remove again. They should be easy to apply, they should impart an attractive shape to the eyelashes and they should retain that shape even after drying. In addition the compositions should be of such low viscosity that they can be satisfactorily applied but on the other hand dry quickly so that the material is not smudged. In addition the product is to be so stable that it can be stored under ambient conditions for a prolonged period of time.

Many different materials have been proposed to resolve all those, partly contradictory tasks. Current mascaras contain a wax component which provides for adhesion to the eyelashes, a film-forming agent which provides for an attractive shape and maintaining that shape and binding agent for setting the viscosity in a suitable range. Mascaras are frequently emulsions in which the continuous phase is formed by water while the discontinuous phase is formed by the wax component. Thus for example U.S. Pat. No. 6,210,692 describes cosmetic emulsions which are suitable inter alia as mascara and which are made up of an aqueous phase and a fat phase, containing for stabilisation purposes two different binding agents, namely a hydrophilic thickening agent and a polysaccharide ether, in which respect the use of such thickening agents which form gels with the aqueous phase is to be avoided. Those emulsions are to be particularly well-suited to caring for and treating dry and sensitive skin.

U.S. Pat. No. 6,325,994 also discloses a cosmetic composition which is suitable in particular as mascara, comprising a lipophilic phase with fat and wax, and a lipophilic polymer, the lipophilic polymer being a special acrylate-methacrylate copolymer. The composition can also be in the form of an emulsion with an aqueous phase. In addition the composition can contain usual additives such as dyes, pigments and fillers. Although that composition is said to be water-resistant and very durable it is however still not satisfactory in every respect.

The literature describes many other similar compositions which usually contain waxes, oils, binding agents and coloring agents and which can also be in the form of oil-in-water emulsions. The known compositions however suffer from at least one of the following disadvantages. Many of the known emulsions are not sufficiently stable for them to be processed in the heated condition. As however a heat treatment is necessary for cosmetic compositions, that requires special treatment steps which are complicated and expensive.

By virtue of the composition thereof it is frequently difficult to incorporate pigments in such a way that they remain stably in the composition without forming lumps or settling. Compositions which enjoy good adhesion to keratinic material are often difficult to remove therefrom again. In particular water-resistant compositions can no longer be removed by aqueous preparations. A major problem which occurs in particular with mascaras is that particles migrate out of the composition and either settle on the skin, which gives rise to unattractive discoloration effects, or, and this is even more serious, they can get into the eye and for example in the case of contact lens wearers discolor the lenses and thus render them useless.

The inventors of the present application therefore set themselves the objects of developing a product which is simple to produce and which can be produced reliably and reproducibly without a special set of machines, into which pigments can be easily incorporated and remain stably contained therein, which is stable even at elevated temperature and which can be heated without separation or decomposition, which at elevated temperature is of such a viscosity that it can be easily introduced into a container, which can be easily applied, which has long-term adhesion, which can extend the length of the eyelashes, give volume to the eyelashes and not be transferred on to lid surfaces, which is water-resistant and tear-resistant, which does not crumble off after drying and which can be easily removed again.

### SUMMARY OF THE INVENTION

The objects are attained by a cosmetic preparation which is in the form of an O/W emulsion and which besides usual cosmetic ingredients contains a wax component, a film-forming agent and a gel component, wherein the gel component contains at least one swollen hydrocolloid.

### DETAILED DESCRIPTION

It was surprisingly found that the use of a swollen hydrocolloid can give the preparation structure and in addition can bind coloring agents and pigments in such a way that they can no longer migrate out of the material so that the applied dried material does not run, even upon coming into contact with water. In addition the swollen hydrocolloid stabilises the structure of the preparation in solid and fluid condition so that no separation of the components of the mixture occurs even at elevated temperature. This has the advantage that it can be heated without problem and can be introduced into containers in the heated condition. Production of the preparation according to the invention is simple and can be easily and reliably effected with conventional available apparatuses.

The cosmetic preparation is intended in particular to be applied to keratinic material, in which respect in particular eyelashes, eyebrows, hair and hair pieces are envisaged. The preparation according to the invention is particularly suitable as mascara.

The most important constituents of the preparation according to the invention, besides the water forming the continuous phase of the emulsion, are a wax component, a film-forming system and at least one gel component.

The wax component comprises at least one wax and preferably additionally at least one oil and/or fat which can respectively be of vegetable, animal, mineral or synthetic origin. In addition the preparation may preferably contain at least one emulsifier and optionally additionally at least one co-emulsifier in order to facilitate processing of the wax component to constitute an emulsion. In order to obtain an aesthetically particularly satisfactory end product, the wax component can additionally contain a vinylpyrrolidone copolymer.

The wax component gives the material consistency and makes the preparation water-resistant. For that purpose the wax component can be made up of fat-like, oil-like and wax-like substances which can be fluid, pasty or solid at ambient temperature. Preferably a combination of at least one wax and at least one oil is used to adjust the optimum consistency.

Preferably at least one wax which is solid at ambient temperature is used. Particularly preferred are such waxes with a dropping point of between 50 and 200° C. Waxes with a dropping point below 50° C. can cause problems in terms of storability and waxes with a dropping point above 200° C. are in part difficult in regard to processing.

The waxes which are usually employed in cosmetics are suitable for the wax component used in accordance with the invention, in particular vegetable waxes such as carnauba wax and candellila wax, ouricurri wax, japan wax, cotton wax, rice wax, flower waxes, hydrogenated jojoba oil; animal waxes such as beeswaxes and modified beeswaxes, inter alia Cera bellina, lanolin waxes and insect waxes; mineral waxes such as paraffin waxes, microcrystalline waxes, montan waxes and ozocerite, as well as synthetic waxes such as Fischer-Tropsch waxes, wax polymer hybrids, polyethylene waxes, silicone waxes and azelaic acid dioleyl esters, azelaic acid dibehenyl esters, behenyloleate, cetyl palmitate and mixtures of all the stated waxes. In a particularly preferred feature animal waxes and synthetic waxes as well as in particular mixtures thereof are used.

At least one fat or oil is contained in the wax component in order to adjust the viscosity of the mass and to impart workability to the mass. For that purpose both animal and also vegetable fats and oils can be considered, which can also be used in hydrogenated or modified state. Examples of suitable fats and oils are rapeseed oil, sunflower oil, sesame oil, groundnut oil, thistle oil, coconut oil, hydrogenated coconut oil, castor oil, hydrogenated castor oil, beef tallow, isopropylmyristate, isopropylpalmitate, isopropylstearate, isobutylstearate, isostearylisostearate, oleyloleate, jojoba oil, caprylic/capric triglyceride and similar synthetic triglyerides, paraffin oil, polybutene, squalane, squalene and mixtures thereof, synthetic esters, silicone oils, in which respect both volatile and also non-volatile oils are considered here, in particular dimethicone and cyclomethicone as well as volatile and non-volatile isoparaffins. Mixtures of those constituents are preferred. Thus frequently both a non-volatile fat or oil and also a volatile oil are contained in order to provide different properties. In an embodiment the wax component contains volatile ingredients, in particular synthetic esters, silicone oils, dimethicone and cyclomethicone and isoparaffins in a concentration of up to 20%.

As mentioned preferably mixtures of waxes, fats and oils are used, wherein the respectively selected substances are employed in such amounts that the desired properties such as texture and viscosity are achieved. The respective amounts and mixtures to be used are known to the man skilled in the art and do not need to be discussed in greater detail here.

In addition the wax component may also have further ingredients which are known per se to the man skilled in the art and which influence properties such as stability, viscosity, workability and durability. Thus for example it may contain sugar derivatives.

Preferably the wax component is used in such a proportion that it forms between 0 and 50%, preferably between 15 and 30% of the finished composition, wherein the percentage information, like all such information in the description unless otherwise specified, relates to weight. Within the wax component preferably the proportion of the wax or waxes is between 20 and 100% and is particularly preferably between 70 and 95%.

The fat or oil is usually added in such a proportion that the consistency of the mass is in the desired range. Normally the proportion of fats and oils in the wax component, depending on the materials used, is between 20 and 80%, wherein the lower range is preferred.

In addition, as stated above, the wax component may also contain emulsifiers and possibly additionally co-emulsifiers. They promote emulsion formation and homogeneity and stabilise the emulsion formed. The selection of those ingredients is not critical, they must however involve the properties required for cosmetic compositions, in particular they may not cause irritation and they may not be toxic. All ingredients which are usually employed in cosmetics and which are known to the man skilled in the art can be used here. Examples are anion-active, cation-active, non-ionogenic emulsifiers and amphotensides, as well as polymer tensides which can be made up for example from copolymers, block copolymers and graft polymers, silicone tensides and polymer silicone tensides, silicone copolymers, PEG derivatives of natural fatty acids and betaines.

The emulsifiers and co-emulsifiers are added to the wax component in the usual amounts; if present the preferred amount (in relation to the wax component) is between about 0.5 and 40%. The optimum amount depends inter alia on the nature and amount of the wax component and the nature of the emulsifiers used and can be easily established by routine tests. Particularly good results are obtained with a proportion in the range of between 10 and 25%.

The second essential component of the cosmetic preparation according to the invention is a film-forming system. The film-forming system is usually formed by a polymer or a plurality of polymers which is or are in dispersion or dissolved in a medium, wherein the polymer or polymers form a film upon removal of the medium, that is to say the solvent or dispersing agent. The solid content, depending on the respective amount of polymer and medium, is usually between 10 and 70%, preferably between 20 and 40%. In the present application any system comprising film-forming agent and medium (also referred to as solubilising agent, solvent or dispersing agent) is referred to generally as a dispersion in order to exclude obscurities as the transitions between a solution and a dispersion are fluid in the field of polymers. The expression ‘dispersion’ therefore also includes solutions. Also included are systems in which the film-forming polymer was solubilised for example by the addition of an acid or a base. The medium is preferably water.

Film-forming polymers are known per se and all polymers which are usually employed for cosmetic preparations can be used. The film-forming agent, together with the wax component, leads to a very durable coating for the keratinic material, for example for eyelashes or eyebrows or hair.

The film-forming polymer can be a polycondensate, a radically produced polymer or a polymer of natural origin. Film-forming systems which are particularly suitable for the preparation according to the invention contain anionic, cationic, non-ionic and/or amphoteric polymers from the class of polyurethanes, polyureas, polyesters and polyethers as well as derivatives thereof, PVP, polyamides, vinyl, acrylic and methacrylic polymers and copolymers, epoxy esters, silicone polymers and copolymers and hybrid polymers formed from the above-mentioned polymers. The film-forming system is usually in the form of a dispersion, aqueous systems being preferred.

Particularly preferred film-forming agents are polyurethanes and polyurethane copolymers such as polyurethane, for example polyurethane-acrylic acid copolymers, polyurethane-polyvinylpyrrolidones, polyester-polyurethanes, polyether-polyurethanes, polyureas, polyester amides, polyesters with fatty chain, polyamides, epoxy ester resins and/or polypropylene glycol-maleic ester copolymers. Many of those polymers are commercially available. A further preferred group are acryl polymers, acryl/styrene copolymers, acryl/vinyl copolymers and acryl/silicone copolymers as well as combinations of nitrocellulose and acryl polymer. Hybrid polymers can also be used according to the invention. Polymers of natural origin which can possibly also occur in modified form such as for example polysaccharide and cellulose derivatives are suitable. A mixture of two or more of the listed polymers can also be used, in which case the mixture is formed in such a way that the desired properties are achieved. The products approved by the CTFA, in particular polyurethane-1 through polyurethane-13, are also particularly suitable.

The film-forming system is used in an amount which gives the desired effect. Amounts in the range of between about 5 and 95% are suitable. In order to achieve particularly advantageous properties, for example in relation to durability and adhesion, amounts in the range of between 5 and 60%, preferably between 20 and 35%, are used, the amount in an individual case depending on the polymer used and the proportion and nature of the wax component and the gel component. The thicker the structure formed by the gel component and the thicker the film formed in each case, the correspondingly less can be the amount of film-forming system. The optimum amount can be easily found by the man skilled in the art. In this respect the indication of amount relates to the system used, that is to say the amount of film-forming polymers and solubilising agent.

The component, which is essential to the invention, of the cosmetic preparation is a gel component which must be included in order to achieve the advantageous properties. It was found that the use of a swollen hydrocolloid as the gel component has a structure-forming action and stabilises the structure in such a way that the composition, after drying, forms a durable, water-resistant film on keratinic material and, together with the film-forming agent and the wax component, retains all ingredients in the composition in such a way that they are not leached out, migrate out or bleed out. Therefore the gel component provides a material which is ideally suited to taking up and retaining coloring agents and pigments and also further ingredients.

The gel component is formed from at least one swollen hydrocolloid, swelling preferably being effected with water. It was established that a hydrocolloid in the swollen condition spreads the desired lattice which then, in combination with the film-forming system, can take up and stabilise other ingredients. In connection with the present invention the term hydrocolloids is to be used to identify such natural and synthetic polymers which in solvents, in particular aqueous systems, form gels or viscous, in particular highly viscous solutions. Examples are in particular celluloses and cellulose derivatives, starch and starch derivatives, alginates, carrageens, pectins, tragacanth and gums as well as polyvinyl alcohol and polyvinyl pyrrolidone and also dextran. Although those substances in powder form are in part already known for use as thickening agents in cosmetics, it was hitherto not recognised that in the swollen condition they can impart particularly advantageous properties to a cosmetic preparation. It was now surprisingly found that those hydrocolloids in the swollen condition impart structure, stability and volume to the preparation according to the invention. The use of the swollen hydrocolloid according to the invention can even provide that the volume, of the preparation scarcely decreases or does not decrease, after the drying operation. As a result the preparation according to the invention, after drying, also makes the eyelashes longer and fuller and enhances the aesthetic impression.

It is essential therefore that the hydrocolloid is used in the swollen condition for production of the preparation according to the invention and it is not just caused to swell in the preparation. Particularly good properties are achieved if the hydrocolloid is swollen in heated water, in particular in water at a temperature of between 40 and 100° C. Usually, the hydrocolloid is mixed with the water, for example with agitation, and is then preferably allowed to cool prior to further processing at least to about 40° C., particularly preferably to ambient temperature. Swelling causes the physical properties of the hydrocolloid to be irreversibly changed, which affords the desired structure. The pre-swollen hydrocolloid produced in that way is stable and storable and therefore can also be stored prior to further processing for some time, for example some hours, or even still longer periods of time.

It is assumed that swelling causes the water binding capacity to increase so that the finished composition can then later bind or hold more water, which inter alia leads to the advantageous properties.

A preferred feature therefore involves using a hydrocolloid with a water binding capacity of between 100 and 600%, preferably between 200 and 500% and in particular between 350 and 430%. The water binding capacity of the hydrocolloid can be determined using per se known methods, for example with Karl-Fischer titration, or by a dry residue determination procedure.

It was further found that a hydrocolloid with a high swelling capacity at 80° C. is particularly advantageous. Preferred hydrocolloids therefore are those whose swelling capacity is in a range of between 250 and 500%, measured at 80° C. The swelling capacity can be determined in per se known manner, for example by a procedure whereby the hydrocolloid is heated in water and, after homogeneously swelling up (that is to say without lump formation), it is cooled with agitation to ambient temperature, the supernatent water is decanted and the water absorbed is determined using the Karl-Fischer method or on a dry weighing device.

A particularly advantageous swelling capacity and a particularly high level of water binding capacity were established in the case of modified starch which is therefore preferably employed. Examples of suitable starches are vegetable starches, in particular cereal starches such as wheat, corn and rice starches, wherein rice starch in modified form is particularly preferred.

In a preferred embodiment the hydrocolloid used is starch or a starch derivative or derivatised or modified starch, in particular rice starch. Particularly advantageous properties can be obtained with hydroxyalkyl- or dimethylimidazolidinone rice starch.

The swollen hydrocolloid, by virtue of its stability, can be stored for a prolonged period of time before it is subjected to further processing for production of the cosmetic preparation. In order to prevent the swollen hydrocolloid becoming bacterially contaminated, a preserving agent can be added to the system of hydrocolloid and water. Preserving agents for such systems are well known to the man skilled in the art and those which are suitable for foodstuffs and cosmetics can be used here.

In addition a dispersing additive can also be added to the system. It was found that a combination of preserving agent and dispersing additive synergistically increases the germicidal effect. Benzyl alcohol and the esters thereof, parabens and salts thereof, phenylethyl alcohol, IPBC, formaldehyde cleaving agents such as diimidazolidinyl urea, organic aromatic acids, phenoxyethanol and others can be named as suitable preserving agents. Examples of dispersing additives are PEG derivatives, non-ionic tensides, block polymers, esters of organic poly-acids, soaps of polyvalent metal salts, amino acids or esters thereof, siliconyl acrylate copolymers and other known wetting agents.

In addition it is possible to use antioxidants such as tocopherol, NDGA, sesame oil, γ-oryzanol, rosemary oil, BHT and other agents used for cosmetics, for stabilisation of the preparation. If necessary microbiological stability can be guaranteed by the addition of preserving agents as are usual for cosmetics.

The proportion of the gel component (that is to say hydrocolloid in swollen condition) in the composition according to the invention can vary in a wide range. A preferred proportion is a proportion of between 3 and 50%, particularly preferably between 3 and 20%. That proportion relates to the weight of swollen hydrocolloid, that is to say hydrocolloid including the absorbed proportion of liquid, in relation to the composition. Below a proportion of 3% the effect is no longer optimal and with a proportion of more than 50% viscosity and structure of the composition can be adversely affected. The proportion of the hydrocolloid in the swollen gel component can also vary within wide limits depending on the nature of the hydrocolloid and can be for example between 5 and 90% depending on the respective swelling capacity.

The gel component of the preparation according to the invention may further have a second gel of a natural polymer, preferably a hydrocolloid, in a monovalent and/or polyvalent alcohol. The second ‘alcoholic’ gel serves to stabilise the structure and to influence the rheological properties. In addition it can influence the drying properties of the preparation. The alcoholic gel component is not absolutely necessary but it is included in preferred embodiments. The polymer of the second gel can be identical to the hydrocolloid of the first gel but is preferably different therefrom.

The second gel used is preferably cellulose derivatives which are soluble in water and alcohol or in mixtures of water and alcohol, in particular carboxymethylcellulose, hydroxymethylcellulose, hydroxyethylcellulose or hydroxypropylcellulose, hydroxypropylcellulose being particularly preferred. The cellulose derivative is so dissolved or dispersed in the alcohol that a gel is formed. Preferably a mixture comprising one or more monovalent alcohols with a chain length of C₂-C₄ and a polyvalent alcohol with a chain length of between C₂ and C₆ and with at least two hydroxyl groups is used for that purpose. Examples of suitable monovalent alcohols are ethanol, n-propanol, i-propanol, n-butanol, i-butanol, ethanol being preferred. Examples of polyvalent alcohols are propane diols such as propane-1,2-diol, dipropane diol, butane diols such as butane-1,2-diol, butane-1,3-diol, butane-1,4-diol, glycerine, diglycerine, triglycerine, diethylene glycols, amyl alcohol, hexane diols, such as hexane-1,2-diol, hexane-1,3-diol, pentaerythritol, sorbitol, xylitol, mannitol and alditol. The polyvalent alcohol is preferably propane-1,2-diol or butane-1,3-diol. Preferably the cellulose derivative is firstly made into a suspension in the polyvalent alcohol and then the monovalent alcohol is added until the desired viscosity is reached. In that situation a highly viscous gel is formed, which can then be mixed together with the further components of the preparation according to the invention.

If an alcoholic gel is used in the preparation according to the invention, the amount thereof depends on the desired viscosity as well as the drying properties. An amount of between 0 and 10% (polymer+alcohols) has proven to be appropriate. In that case the proportion of the polymer is in a range of between 0.5 and 20% of the gel. If the preparation dries very slowly the proportion of the alcoholic gel or the proportion of alcohol in the gel respectively can be increased.

In addition the preparation according to the invention may also contain further usual cosmetic ingredients which influence specific desired properties. An important ingredient for most cosmetic products are coloring agents or pigments in order to produce a desirable color. It has been found that the preparation according to the invention is suitable for stabilising both organic coloring agents and also pigments in such a way that they are not leached out. All substances which are known for cosmetics can be used as pigments or coloring agents. The only prerequisite is that the substances are not toxic and irritating. The pigments are preferably used in micronised form. Examples of pigments are iron oxides, ultramarine, chromium oxide green, chromium hydroxide green, carbon black, titanium dioxide, zinc oxide, barium sulfate, talcum and kaolin. Examples of organic coloring agents are lakes such as aluminum, barium, calcium, potassium, strontium and zinc lakes, carmine and other coloring substances which are well known to the man skilled in the art. In addition the preparation may also contain pearl gloss pigments. The particulate ingredients are used in the proportion which is usually employed for cosmetics, the amount being selected in dependence on the nature of the particle phase and the desired effect. Preferably the proportion of the particulate ingredients is in a range of up to 30%, particularly preferably between 2 and 20%.

In addition the preparation according to the invention may contain fillers and particulate substances which give a desired structure to the mass. Those fillers and particles are also stabilised by the preparation according to the invention so that they do not settle. Examples are talcum, kaolin, bentonite, hectorite, montmorillonite, cerium oxide, silicon dioxide, boronitride, nylon powder, polyethylene powder, polypropylene powder, silk powder and mixtures thereof, polyvalent metal soaps, non-swellable starches, fruit fibers, natural and synthetic exfoliation substances, sand, bran products, stone powder from stone fruits, algae derivatives, thermosetting, thermoplastic and elastomer powders and mixtures and hybrids of the listed constituents.

In order to make the preparation velvety and glossy, it is also possible to add plasticisers. Examples thereof are polyvalent alcohols and esters thereof such as glycerine, diglycerine, triglycerine, diethylene glycols, amyl alcohol, hexane diols, pentaerythritol, sorbitol, xylitol, mannitol and alditol. Saccharose acetate isobutyrate, laureth-2-benzoate, ethylhexylsebacate, citric acid esters such as tributylcitrate, synthetic short-chain esters, pentaerythritol esters and oligopentaerythritol esters.

If the preparation according to the invention is used as mascara, usually such pigments and coloring agents are used, which result in the colors that are usually desired, namely black, blue, brown and gray. It is however equally possible to produce differently colored preparations in order for example to color the tips of the hair or the tips of the eyelashes in a different color from the rest of the hair or eyelashes.

The coloring ingredients, that is to say pigments and coloring agents, do not necessarily have to be included in the preparation according to the invention. An embodiment is also possible in which coloring and structure-imparting constituents are separate and firstly the preparation according to the invention without coloring ingredients is applied to the keratinic material and then a formulation of the coloring agents or pigments is applied.

The pH-value of the preparation according to the invention should preferably be in the neutral to weakly basic range as many of the film-forming agents which are usually employed in cosmetics can already precipitate at a weakly acid pH-value. Therefore to adjust or buffer the pH-value the preparation possibly also contains basic agents and/or buffering agents, for example NaOH, amines and conventional buffers. Preferably the pH-value of the preparation is so adjusted that it is in a range of between 6.5 and 8.8, particularly preferably between 6.8 and 7.5.

In accordance with the invention therefore there is provided a preparation which is in the form of an emulsion and which combines particularly advantageous properties. The preparation can be easily produced, it leads to stable products, it can be easily subjected to further processing as it is stable in the hot condition and it produces a film which adheres for a long period of time without liberation of the ingredients contained therein. The film causes the keratin fibers to which it is applied to look full and ‘curls’ them in an attractive and long-lasting manner. At the desired time the preparation can also be easily removed again by applying water, optionally with wetting agent, which leads to swelling of the film whereby it can be easily taken off again. The specific combination of wax component, film-forming agent and gel component forms a network or lattice which adheres durably to the keratin fiber so that the result is a homogeneous, bulky film which adheres firmly.

A further subject of the invention is a process for the production of that preparation.

To produce the cosmetic preparation according to the invention the aqueous gel component is firstly prepared by the hydrocolloid being caused to swell with water. For that purpose preferably heated water which is advantageously at a temperature of between 40° C. and 100° C., preferably between about 65 and 90° C., is brought into contact with the colloid with agitation and then thereafter possibly cooled and allowed to swell. A preserving agent and/or a dispersing additive can also optionally be added to the mixture. The swollen gel is stable and can be stored even over prolonged periods of time so that it does not have to be subjected to further processing immediately.

The wax used for the wax component is melted, optionally together with the fat and/or oil, and preferably raised to a temperature in the range of between 50 and 100° C., preferably between 65 and 85° C. The amount of water necessary to produce the emulsion and which can contain emulsifiers and co-emulsifiers is preferably also heated and then mixed with the wax component. The procedure then involves adding to the resulting O/W emulsion, the swollen aqueous hydrocolloid, optionally the alcoholic gel and the dispersion of the film-forming agent. The mixing operation is effected in each case until a homogeneous dispersion or emulsion is produced. The particulate constituents, in particular pigments and fillers, can then also optionally be added by mixing, optionally with auxiliary substances which improve compatibility.

The mixture is then agitated until it is homogeneous and is then cooled with agitation. It is then optionally possible to add to that mixture, either while still in the hot condition or, if sensitive additives are involved, during the cooling operation, the active substances and additives which are usually added for cosmetic purposes such as perfumes, plasticisers, gloss substances and so forth.

The preparation obtained is then introduced into packaging containers for filling purposes. As the preparation according to the invention is distinguished by particular stability, it can also be packaged in the heated condition, in which case ‘cold agitation’ is not necessary. That is advantageous on the one hand for reasons of hygiene and on the other hand as flowability is generally better in the hot condition.

For specific embodiments it has proven to be advantageous for a part of the film-forming agent to be added to the wax component, which results in particular aesthetic products. A vinylpyrrolidone copolymer is used in particular as the film-forming agent for the wax component, for this embodiment.

The process according to the invention makes it possible for the preparation to be produced easily, reproducibly and in large numbers. As, by virtue of its stability, the preparation can be processed at elevated temperature, it has advantages over known products, both in regard to hygiene and also in regard to process engineering.

The preparation according to the invention is particularly well suited to coating keratinic materials. It is therefore preferably used for coloring and/or shaping eyelashes, eyebrows, hair, hair pieces, beards and other keratinic constituents. The preparation according to the invention is particularly suitable as mascara as it adheres firmly to eyelashes, it does not bleed out, it does not crumble away and it remains on the eyelashes for a long time and durably until it is removed again.

A further subject of the invention is therefore the use of the preparation according to the invention for coloring and shaping keratinic material and in particular for the production of mascaras.

The invention is described by the following Example without being restricted thereto.

### EXAMPLE 1

A mascara material was produced of the following composition (amounts respectively given in parts by weight, with the ingredients being denoted by the INCI/CTFA names):

| | |
|---|---|
| 2.3 | Cera carnauba/Copernicia conifera (carnauba) wax |
| 9.2 | Cera alba/beeswax |
| 4.0 | Sun flower oil |
| 1.7 | Stearic acid |
| 1.7 | Glyceryl stearate |
| 0.2 | Antioxidant |
| 48 | Aqua |
| 0.5 | Sodium lauryl sulfate |
| 3.4 | Iron oxides |
| 3.4 | Mica |
| 1.8 | Diazolidinyl rice starch * |
| 0.8 | Almond oil PEG-6 ester |
| 0.2 | Preservative |
| 3 | Ethylene glycol |
| 19.8 | Acrylates copolymer |

* (The amount by weight of the hydrocolloid (diazolidinyl rice starch) relates to the weight prior to swelling).
The specified constituents were used to produce an emulsion as described above which, after application, adhered for a long time.

1-29. (canceled)

30. A cosmetic preparation in the form of an O/W emulsion which besides usual cosmetic ingredients contains a wax component, a film-forming system and at least one gel component, wherein the gel component has at least one swollen hydrocolloid.

31. A cosmetic preparation as set forth in claim 30, wherein the wax component comprises one or more waxes which are solid at ambient temperature and which have a dropping point of between 50 and 200° C.

32. A cosmetic preparation as set forth in claim 31, wherein the wax component further comprises a combination of emulsifier and co-emulsifier.

33. A cosmetic preparation as set forth in claim 32, wherein the wax component further comprises volatile constituents selected from the group consisting of synthetic esters, silicone oils, dimethicone, cyclomethicone, isoparaffins and mixtures thereof in a concentration of up to 20%.

34. A cosmetic preparation as set forth in claim 30, wherein the film-forming system comprises a film-forming polymer which is dispersed and/or dissolved in a medium.

35. A cosmetic preparation as set forth in claim 34, wherein the film-forming system is selected from the group consisting of an anionic, cationic, non-ionic or amphoteric polyurethane, polyurethane-acrylic acid polymer, polyurethane-polyvinylpyrrolidone, polyester-polyurethane, polyether-polyurethane, polyurea, polyester, polyester amide, polyester with fatty chain, polyamide, epoxy ester resin, acryl polymer, vinyl polymer, acryl-silicone copolymer, nitrocellulose/acrylic copolymer, polymer of natural origin, hybrid polymer, polycondensation product, silicone copolymer and mixtures thereof.

36. A cosmetic preparation as set forth in claim 30, wherein the hydrocolloid comprises a water-soluble, polymer which swells in water and forms a gel or a highly viscous solution.

37. A cosmetic preparation as set forth in claim 30, wherein the hydrocolloid is selected from the group consisting of alginates, carrageens, pectins, tragacanth, gums, celluloses, starches, polyvinyl alcohol, polyvinylpyrrolidone and mixtures thereof.

38. A cosmetic preparation as set forth in claim 30, wherein it contains an aqueous gel and an alcoholic gel.

39. A cosmetic preparation as set forth in claim 30, wherein the aqueous gel comprises a starch derivative which is swollen in water.

40. A cosmetic preparation as set forth in claim 30, wherein the gel is an alcoholic gel which comprises a cellulose derivative which is swollen with at least one of a monovalent and polyvalent alcohol, wherein the monovalent alcohol is a C₂-C₄ alcohol and the polyvalent alcohol is a C₂-C₆ alcohol.

41. A cosmetic preparation as set forth in claim 40, wherein the cellulose derivative is selected from the group consisting of hydroxymethylcellulose, hydroxyethylcellulose, hydroxypropylcellulose, carboxymethylcellulose; hydroxyalkyl starch, dimethylimidazolidinone starch and mixtures thereof.

42. A cosmetic preparation as set forth in claim 30, wherein the gel comprises at least one preserving agent, at least one dispersing additive, and optionally at least one plasticiser.

43. A cosmetic preparation as set forth in claim 41, wherein the swollen gel is contained in a proportion of between 3 and 50%.

44. A cosmetic preparation as set forth in claim 30, wherein the hydrocolloid has a water absorption capacity in a range of between 100 and 600%.

45. A cosmetic preparation as set forth in claim 30, wherein the hydrocolloid has a water absorption capacity in a range of between 200 and 500%.

46. A cosmetic preparation as set forth in claim 30, wherein the preparation further comprises a particle phase.

47. A cosmetic preparation as set forth in claim 46, wherein the particle phase is selected from the group consisting of inorganic pigments, organic pigments, fillers and mixtures thereof.

48. A cosmetic preparation as set forth in claim 47, wherein pigment contains at least one of iron oxides, ultramarine, chromium oxide green, chromium hydroxide green, carbon black, titanium dioxide, zinc oxide, barium sulfate, carmine, lakes of organic coloring agents, aluminum, barium, calcium, potassium, strontium zinc lake, talcum and kaolin.

49. A cosmetic preparation as set forth in claim 47, wherein the fillers are selected from the group consisting of talcum, kaolin, bentonite, hectorite, montmorillonite, cerium oxide, silicon dioxide, ornitrite, nylon powder, polyethylene powder, polypropylene powder, silk powder, polyvalent metal soaps, non-swellable starches, fruit fibers, exfoliation substances, sand, bran products, stone powder from stone fruits, algae derivatives, thermosetting, thermoplastic, elastomer powder, and mixtures thereof.

50. A cosmetic preparation as set forth in claim 47, wherein the particle phase is present in an amount up to 30%.

51. A cosmetic preparation as set forth in claim 30, wherein the wax component is included in a proportion of between 15 and 30%, the film-forming system is included in a proportion of between 5 and 60%, the gel component is included in a proportion of between 3 and 50%, in each case with respect to the weight of the total composition, the balance being water and optionally cosmetic additives.

52. A cosmetic preparation as set forth in claim 51, wherein the cosmetic additives are selected from the group consisting of preserving agent, perfumes, extracts, protein hydrolysate, silk powder, and mixtures thereof.

53. A process for the production of a gel-bearing cosmetic preparation comprises swelling a hydrocolloid in water, melting a wax component, forming an emulsion from the wax component by mixing the wax component with water and an emulsifier, adding the swollen hydrocolloid to the emulsion with a film forming system and agitating until homogeneous.

54. A process as set forth in claim 53, further including adding a particle phase to the mixture prior to agitating.

55. A process as set forth in claim 53, including adding to the mixture an alcoholic gel comprising at least one monovalent alcohol, at least one polyvalent alcohol and a gel-forming agent.

56. A process as set forth in claim 53, wherein the mixture is adjusted to a ph value of between 6.5 and 8.8.

57. A process as set forth in claim 53, including shaping and/or coloring keratinic material.

58. A process as set forth in claim 53, including producing mascara.

59. A process as set forth in claim 57, wherein the keratinic material eyelashes, eyebrows, hair on the head, hair pieces and beard hair are colored.
